(19) United States
(12) Patent Application Publication
(10) Pub. No.: US 2012/0196297 A1
(43) Pub. Date: Aug. 2, 2012

(54) MASS TAGS FOR MASS SPECTROMETRIC ANALYSIS OF IMMUNOGLOBULINS

(76) Inventor: Richard A. Yost, Gainesville, FL (US)

(21) Appl. No.: 13/383,603

(22) PCT Filed: Jul. 14, 2010
(86) PCT No.: PCT/US10/41936

§ 371 Date: Apr. 19, 2012

Related U.S. Application Data
(60) Provisional application No. 61/225,273, filed on Jul. 14, 2009.

(51) Int. Cl. G01N 33/566; C07K 16/00; C07K 19/00; C07K 16/42

(52) U.S. Cl. 435/7.1; 436/501; 530/391.3; 530/403

(57) ABSTRACT

The present invention is a method for the characterization or detection of one or more antibodies in a sample. The method comprises obtaining a tagged antigen comprising an antigen and a mass tag attached to the antigen. The tagged antigen has specificity for the antibody. In addition, the method comprises combining the tagged antigen with the antibody to form a tagged antigen-antibody complex. Further, the method comprises cleaving the mass tag from the tagged antigen-antibody complex. Thereafter, the method comprises analyzing the mass tag via a mass spectrometer to determine the presence of the mass tag in the sample and correlating the presence of the mass tag with a presence of the antibody in the sample.

### RELATED APPLICATIONS

This application claims priority to U.S. Ser. No. 61/225,273, filed Jul. 14, 2009, which is incorporated herein by reference in its entirety.

### FIELD OF THE INVENTION

The present invention relates to methods for the characterization of immunoglobulins by mass spectrometry, and more particularly to vastly simplified methods for the characterization of immunoglobulins by mass spectrometry, wherein mass tags are attached to antigens that will complex with corresponding target immunoglobulins. Thereafter, the mass tags are cleaved from the immunoglobulin-antigen complex, and are detected by mass spectrometry. Kits for performing the disclosed methods are also provided.

### BACKGROUND OF THE INVENTION

Mass spectrometry is an analytic technique that separates charged particles by their interaction with an electric or magnetic field to find the relative masses of molecular ions and fragment ions. Mass spectrometry has a number of applications including, but not limited to, determining molecular mass, determining the structure of an unknown substance, confirming the identity and purity of a known substance, and providing data on isotopic abundance.

Generally, mass spectrometers can be divided into three fundamental parts, namely the ionization source, the analyzer, and the detector. Depending on the size of the target molecules, concentration of target compounds, degree of qualitative vs. quantitative information desired, the choice and type of ionization source and analyzer may be selected to qualitatively or quantitatively analyze samples ranging from the smallest of molecules to proteins of >40 kDa. To obtain a selected desired outcome from mass spectrometric analysis, a number of different mass spectrometers have evolved. For example, MALDI-TOF (matrix-assisted laser desorption/ionization-time of flight) mass spectrometers have been developed, which are particularly useful for the analysis of large biomolecules, such as immunoglobulins (˜150 kDa) because they provide a single charge to the biomolecule. Nevertheless, the analysis of large biomolecules poses specific challenges associated with the size of the molecules, such as low sample volatility, limited mass resolution and mass accuracy, poor detector efficiency, sample heterogeneity, the production of complexes, and the like. Additionally, the analysis of large biomolecules by MALDI-TOF mass spectrometry typically cannot be used reliably for quantitative analysis because of ion suppression problems associated with the MALDI ionization process. Accordingly, methods, systems, and kits that would simplify the characterization of large biomolecules, such as immunoglobulins, are needed.

### BRIEF DESCRIPTION OF THE DRAWINGS

FIG. 1 depicts a tagged antigen comprising a mass tag bonded to an antigen in accordance with an aspect of the present invention;

FIG. 2 depicts a tagged antigen complexed with an antibody in accordance with an aspect of the present invention;

FIG. 3 depicts a tagged antigen-antibody complexed captured by a biotin-tagged Protein A/G in accordance with an aspect of the present invention;

FIG. 4 depicts a biotin-tagged complex bound to a magnetic bead in accordance with an aspect of the present invention; and

FIGS. 5A-5C depict the interrelationship of the components used for the characterization of an immunoglobulin in a sample in accordance with an aspect of the present invention.

### DETAILED DESCRIPTION OF THE INVENTION

In accordance with an aspect of the present invention, there is provided a method for the detection of one or more antibodies in a sample. In the method, a tagged antigen is obtained by, for example, attaching a mass-tag to an antigen or otherwise acquiring a tagged antigen product comprising an antigen and a mass tag attached to or otherwise associated with the antigen. The tagged antigen is specific to the antibody. Thereafter, the tagged antigen and the antibody are combined to form a tagged antigen-antibody complex. The mass tag is then cleaved from the tagged antigen-antibody complex. Next, the mass tag is analyzed via mass spectrometry to at least determine the presence of the mass tag in the sample. Lastly, the presence of the mass tag is correlated with the presence of the antibody in the sample. The method sets forth a unique approach to the analysis of antibodies (immunoglobulins), which vastly simplifies and shortens current immunoglobulin assays performed using mass spectrometry.

As used herein, the term “antigen” is used in its broadest context. An “antigen” may be any molecule, cell, virus, or particle. For example, an antigen includes, but is not limited to, a chemical molecule, a peptide molecule, a protein molecule, an RNA molecule, a DNA molecule, a traditional antibody, e.g., two heavy chains and two light chains, a recombinant antibody or fragment, a bacterial cell, a virus particle, a cell, a particle, and a product cross-linking any two or more of the above. The antigen may be in a pure form, or may exist in a mixture. In addition, the antigen may be in a modified form (e.g., modified by chemicals) or can be in an unmodified form. It is understood that it is merely critical that an antibody binds with the antigen and has a degree of selectivity for the antigen. In a specific embodiment, the antigen is a secondary antibody that binds to a primary antibody or primary antibody fragment. The primary antibody or primary antibody fragment is typically one found in a biological sample for a subject. In an alternative embodiment, the antigen is an anti-idiotype antibody, i.e., an antibody that reacts with antigenic determinants of another antibody. In another embodiment, the antigen is a biomarker for a disease or disorder.

As used herein, the term “antibody” or “immunoglobulin” refers to any polypeptide that binds to an antigen. An antibody may include, but is not limited to, for example, a traditional antibody, a fragment of a traditional antibody containing an antigen binding site, a recombinant antibody containing an antigen binding site, a protein which binds to an antigen, and a product obtained by cross-linking any two or more of the above.

As used herein, “mass tag” refers to any chemical moiety or moieties which will bind, preferably temporarily, to an antigen as defined herein either directly or via a linker.

As used herein, the term “mass spectrometry” (MS) refers to methods of filtering, detecting, and measuring ions based on their mass-to-charge ratio, or “m/z.” Generally, one or more molecules of interest are ionized, and the ions are subsequently introduced into a mass spectrometer where, due to a combination of magnetic and electric fields, the ions follow a path in space that is dependent upon mass and charge. See, e.g., U.S. Pat. Nos. 6,204,500, 6,107,623, 6,268,144, 6,124,137 for further explanation.

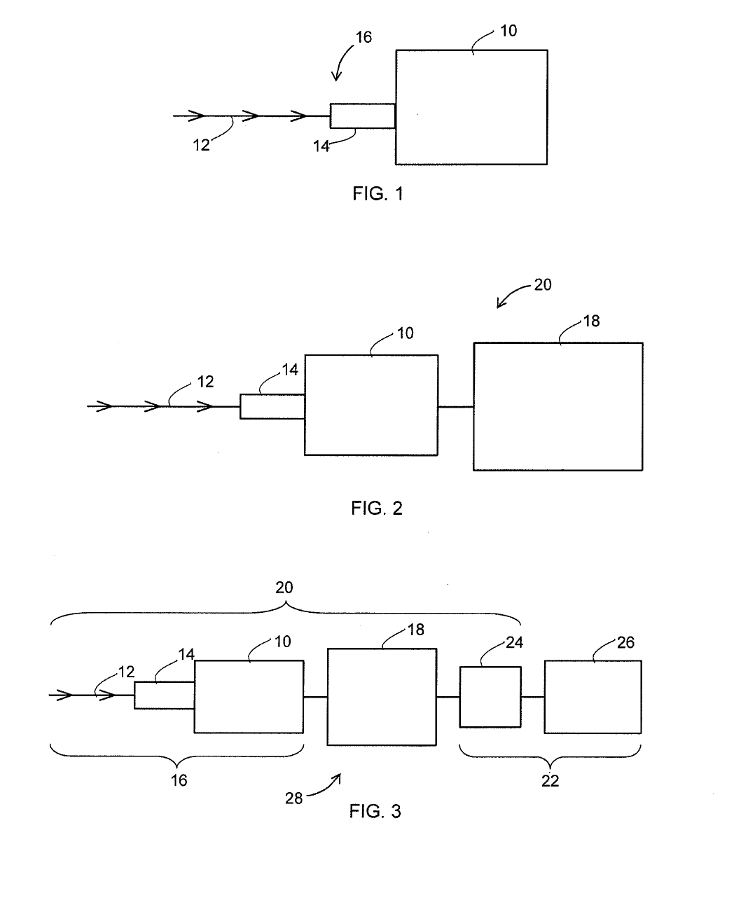

Now referring to the drawings, as shown in FIG. 1, there is shown an exemplary antigen 10 in accordance with the present invention with a mass tag 12 bonded to a corresponding functional group of the antigen 10, typically via covalent bonding (though not required). In one embodiment, the mass tag 12 is directly bonded to the antigen 10 through one or more functional groups on the mass tag 12 that individually or collectively define a linker 14 and corresponding functional groups on the antigen 10. In another embodiment, the mass tag 12 is bonded to the antigen 10 via a linker 14 that is in the form of an intermediate chemical entity having functional groups to bond to or otherwise attach to both the mass tag 12 and the antigen 10. For purposes of illustration only, the linker 14 is shown in FIG. 1 as an intermediate structure between the mass tag 12 and the antigen 10. It is appreciated, however, that the linker 14 may also be integral with the mass tag 12 and may comprise one or more chemical moieties thereon to attach to the antigen 10. The mass tag 12 must be capable of being detected and distinguished from any other mass tag via mass spectrometry. Importantly, the mass tag 12 must also be chemically stable toward all manipulations to which it is subjected, including attachment to and cleavage from the antigen 10, and/or any manipulations of the antibody-antigen complex (once formed) while the tag 12 is attached to the complex. Moreover, the mass tag 12 should not significantly interfere with antigen recognition by the antibody as discussed below.

Because the mass tag 12 is analyzable by mass spectrometry, the mass tag 12 is preferably capable of being ionized. In other words, the mass tag 12 may carry a positive or negative charge under conditions of ionization in the mass spectrometer, and preferably a positive charge. Ideally, the mass tag 12 is selected to provide improved efficiency of ion formation and greater overall sensitivity of detection, especially in comparison to an antibody molecule. Exemplary parameters that can increase the relative sensitivity of an analyte being detected by mass spectrometry are discussed in Sunner, J., et al., Anal. Chem. 60:1300-1307 (1988), for example. Exemplary chemical moieties that will facilitate negative ion formation include, but are not limited to, phenolic hydroxyl, phosphonate, phosphate, tetrazole, sulfonyl urea, perfluoro alcohol, sulfonic acid, and organic acids. Exemplary chemical moieties that will facilitate positive ion formation include amides and aliphatic or aromatic amines. Examples of amine functional groups which give enhanced detectability of mass tags include tertiary and quaternary amines and tertiary amines (including C═N—C groups such as are present in pyridine, including alkyl or aryl tertiary amines). See U.S. Pat. No. 5,240,859; Hess et al., Anal. Biochem. 224:373, 1995; Bures et al., Anal. Biochem. 224:364, 1995.

The identification of the mass tag 12 by mass spectrometry is generally based upon its molecular mass to charge ratio (m/z). In one embodiment, the molecular mass range of the mass tag 12 is from about 100 to 2,000 daltons. In a particular embodiment, the mass tag 12 has a mass of at least about 250 daltons because is more difficult for mass spectrometers to distinguish chemical moieties having parent ions below about 200-250 daltons from background species such as MALDI matrix ions (depending on the instrument), and thus mass tags of the present invention will typically have a mass greater than at least 250 daltons.

In addition, it is relatively difficult to differentiate chemical moieties by mass spectrometry when those moieties incorporate atoms that have more than one isotope in significant abundance. Accordingly, in one embodiment, the mass tag 12 comprises carbon, at least one of hydrogen or fluorine, and at least one of oxygen, nitrogen, sulfur, phosphorus, or iodine. While other atoms may be present in the mass tag 12, their presence typically complicates the analysis of the respective mass spectral data. In one embodiment, the mass tag 12 includes carbon, nitrogen and oxygen atoms, in addition to hydrogen and/or fluorine. In another embodiment, the mass tag is enriched or depleted in a particular isotope.

As mentioned, fluorine is a preferred component of the mass tag 12. In comparison to hydrogen, fluorine is much heavier. Thus, the presence of fluorine atoms rather than hydrogen atoms leads to chemical groups of higher mass, thereby allowing the mass tag 12 to reach and exceed a mass of greater than 250 daltons, which is preferred as indicated above. In addition, the replacement of hydrogen with fluorine provides greater volatility to the mass tag 12, and greater volatility typically enhances sensitivity via mass spectrometric analysis. Accordingly, in one aspect of the present invention, the mass tag 12 comprises a fluorous mass tag. Exemplary fluorous mass tags are commercially available from Fluorous Technologies, Inc., Pittsburgh, Pa. (www.fluorous.com).

For example, a fluorous mass tag may comprise any one or more of the following: N-[(3-perfluorobutyl)propyl]iodoacetamide (C₉H₉F₉INO; FW: 445.10); N-[(3-perfluorohexyl)propyl]iodoacetamide (C₁₁H₉F₁₃INO; FW: 545.09); N-[(3-perfluorooctyl)propyl]iodoacetamide (C₁₃H₉F₁₇INO; FW: 645.10); 2-aminooxy-N-(3-perfluorobutyl-propyl)acetamide (C₉H₁₁F₉N₂O₂; FW: 350.19); 2-Amino oxy-N-(3-perfluorohexyl-propyl)acetamide (C₁₁H₁₁F₁₃N₂O₂; FW: 450.20); 2-Aminooxy-N-(3-perfluorooctyl-propyl)acetamide (C₁₃H₁₁F₁₇H₂O₂; FW: 550.21); 3-(perfluorobutyl)propyl-1-maleimide (C₁₁H₈F₉NO₂; FW: 357.18); 3-(perfluorohexyl)propyl-1-maleimide (C₁₃H₈F₁₃NO₂; FW: 457.21); 3-(perfluorooctyl)propyl-1-maleimide (C₁₅H₈F₁₇NO₂; FW: 557.21); N-Succinimidyl 3-perfluorobutylpropionate (C₁₁H₈F₉NO₄; FW: 389.18); N-Succinimidyl 3-perfluorohexylpropionate (C₁₃H₈F₁₃NO₄; FW: 489.18); N-Succinimidyl perfluorooctylpropionate (C₁₅H₈F₇NO₄; FW: 589.21); 3-perfluorobutyl-propylamine (C₇H₈F₉N; FW: 277.15); 3-perfluorohexyl-propylamine (C₉H₈F₁₃N; FW: 377.15); 3-perfluorobutyl-propylazide (C₇H₆F₉N₃; FW: 303.13); 3-perfluorohexyl-propylazide (C₉H₆F₁₃N₃; FW: 403.16); 3-perfluorooctyl-propylazide (C₁₁H₆F₁₇N₃; FW: 503.18); 1H,1H,2H,2H-Perfluorohexane-1-thiol (C₆H₅F₉S; FW: 280.11); 1H,1H,2H,2H-Perfluorooctane-1-thiol (C₈H₅F₁₃S; FW: 380.13). The above fluorous mass tags are particularly suitable for attaching to carbonyl groups of a target molecule.

For further discussion of exemplary fluorous mass tags for use in the present invention, see Dandapani, S., Recent applications of fluorous separation methods in organic and bioorganic chemistry. QSAR & Combinatorial Science 2006, 25, (8-9), 681-688; Brittain, S. M.; Ficarro, S. B.; Brock, A.; Peters, E. C., Enrichment and analysis of peptide subsets using fluorous affinity tags and mass spectrometry. Nature Biotechnology 2005, 23, (4), 463-468; Go, E. P.; Uritboonthai, W.; Apon, J. V.; Trauger, S. A.; Nordstrom, A.; O'Maille, G.; Brittain, S. M.; Peters, E. C.; Siuzdak, G., Selective Metabolite and Peptide Capture/Mass Detection Using Fluorous Affinity Tags. J. Proteome Res. 2007; 6,(4), 1492-1499; Richard D. Smith, Gordon A. Anderson, Mary S. Lipton, Christophe Masselon, Ljiljana Pas\check{s}a-Tolic', Yufeng Shen, Harold R. Udseth. OMICS: A Journal of Integrative Biology. Jan. 1, 2002, 6(1): 61-90.

The linker 14 that bonds the mass tag 12 to a target antigen 10 may include a direct covalent bond or may comprise one or more organic chemical moieties, which are used to bond the mass tag 12 to the antigen 10. The direct bond or the bonds of the one or more chemical moieties within the linker 14 are cleavable under conditions that allow the mass tag 12 to be cleaved from the linker 14. Optionally, cleavage of the linker 14 may be accomplished rapidly, e.g. in under a minute. Examples of cleavable linkers are well known to those in the art and are commercially available, e.g., from Thermo Fisher Scientific, Rockford, Ill.

In one embodiment, one or more linkers 14 may be bonded to an individual antigen 10 to form a tagged antigen 16. In another embodiment, more than one mass tag 12 may be bonded to a single linker 14 at more than one bonding site on the linker 14. In either case, there may be provided a plurality of the mass tags 12 for a single antigen, thereby increasing specificity and sensitivity for the particular mass tag 12. When the linker(s) 14 is cleaved, the remaining mass tags 12 (and any remaining portion of the linker(s) 14) are analyzed by mass spectrometry to identify the presence and/or amount of an antibody in a solution as will be explained below.

As with the mass tag 12, the linker 14 must also be stable toward all manipulations to which it is subjected, with the exception of the conditions that will allow cleavage and release of the mass tag 12 from the linker 14. Thus, the linker 14 is stable during attachment of the mass tag 12 to the linker 14, attachment of the mass tag 12 by the linker 14 to the antigen 10, and/or any manipulations of the antigen 10 or antibody-antigen complex while the mass tag 12 is attached. For an extensive discussion of linkers that are labile to actinic radiation (e.g., photolysis), as well as acid, base and other cleavage conditions, see for example, Lloyd-Williams, P., et al., Convergent Solid-Phase Peptide Synthesis, Tetrahedron Report No. 347, 49(48):11065-11133 (1993).

In one embodiment, the linker 14 is one that is cleavable by pH adjustment or the addition of a reducing agent as is known in the art. Several acid-labile linker moieties that have been developed for solid phase peptide synthesis are useful for linking one or more mass tags 12 to an antigen 10 in the present invention. Exemplary linkers include 4-hydroxymethylphenoxyacetic acid and 4-(4-hydroxymethyl-3-methoxyphenoxy)butyric acid, which are each commercially available from Advanced ChemTech™ (Louisville, Ky.). Each of these linkers may be attached to the mass tag 12 via an ester linkage or an amide linkage, for example, and cleaved under suitable pH conditions. In another embodiment, the linker 14 may comprise any one or more of disulfide bonds, acid or base labile groups, including among others, silyl ethers, carbamates, oxyesters, ethers, polyethers, diamines, ether diamines, polyether diamines, polythioethers, disulfides, alkyl or alkenyl chains (straight chain or branched and portions of which may be cyclic) aryl, diaryl or alkyl-aryl groups, amides, polyamides, and esters.

In another embodiment, the linker 14 may be an enzymatically cleavable linker. Enzymatically cleavable linkers include, but are not limited to, protease-sensitive amides or esters, beta-lactamase-sensitive beta-lactam analogs and linkers that are nuclease-cleavable, or glycosidase-cleavable.

In yet another embodiment, the linker 14 may be one that is cleavable by photolysis. Exemplary photocleavable linkers 14 include nitrophenyl glycine esters, exo- and endo-2-benzonorborneyl chlorides, methane sulfonates, and 3-amino-3(2-nitrophenyl) propionic acid. Two further exemplary photolabile linkers that have been reported in the literature are 4-(4-(1-Fmoc-amino)ethyl)-2-methoxy-5-nitrophenoxy)butanoic acid (Holmes and Jones, J. Org. Chem. 60:2318-2319, 1995) and 3-(Fmoc-amino)-3-(2-nitrophenyl)propionic acid (Brown et al., Molecular Diversity 1:4-12, 1995). These linkers may be attached via a carboxylic acid to an amine on the target molecule, for example.

Additional types of linkers are described in, e.g., Backes and Ellman (1997) Curr. Opin. Chem. Biol. 1:86-93, Backes et al. (1996), J. Amer. Chem. Soc. 118:3055-3056, Backes and Ellman (1994), J. Amer. Chem. Soc. 116:11171-11172, Hoffmann and Frank (1994), Tetrahedron Lett. 35:7763-7766, Kocis et al. (1993), Tetrahedron Lett. 34:7251-7252, and Plunkett and Ellman (1995), J. Org. Chem. 60:6006-6007.

Any suitable method known in the art may be utilized to bond the mass tag 12 to the respective antigen 10 and to form a tagged-antigen product 16 as was shown in FIG. 1. For example, a solution containing a plurality of the mass tags 12 may be combined with a solution containing one or more antigens 10. As described above, the antigen 10 may be any molecule, cell, virus, or particle, such as a chemical molecule, a peptide molecule, a protein molecule, an RNA molecule, a DNA molecule, a traditional antibody. In a particular embodiment, the antigen 10 comprises an anti-IgG antibody. Once combined, the solution comprising a plurality of mass tags 12 and antigen 10 may be buffered to a suitable pH and allowed to incubate for a suitable time period, e.g. from 1 to 24 hours to attach one or more mass tags 12 to a respective antigen 10.

In one embodiment, a single mass tag 12 is bonded to a single respective antigen 10. In yet another embodiment, as mentioned above, more than one mass tag 12 may be bonded to a respective antigen 10 to thereby increase sensitivity for the target molecule (the corresponding antibody) when analyzed via mass spectrometry. In one embodiment, this may be accomplished by constructing a plurality of the mass tags 12 on a suitable scaffold to attach a plurality of the mass tags 12 to a respective antigen 10 via linkers 14 such that upon cleavage of the linkers 14 bonded to the antigen 10 (which is also complexed with an antibody), multiple individual mass tags 12 are freed. Each of the mass tags 12 will produce a detectable signal so that each antibody can be marked by multiple detectable signals as will be discussed below.

Once the antigen 10 is tagged, a sample comprising one or more immunoglobulins 18 (antibodies 18) is added to a solution containing the tagged antigens 18 to form a tagged antigen-antibody complex 20. The complex 20 comprises a mass tag 12 bonded to an antigen 10, which is complexed with an antibody 18 as shown in FIG. 3. Immunoglobulins (antibodies) are a group of structurally related proteins composed of heavy and light chains. These proteins are categorized as IgM, IgG, IgD, IgE, and IgA depending upon the characteristics of the constant regions of their heavy chains (designated μ, γ, δ, ε, and α, respectively). The variable regions of the heavy chains along with the variable regions of the light chains determine the molecular (antibody) specificity of the complete molecule. In one embodiment, the immunoglobulin 20 comprises IgG. The immunoglobulin-containing sample may be of any suitable size, such as 100 μL and may be maintained at a suitable pH to maintain the integrity of the immunoglobulins 18 therein. The solution comprising the tagged antigens 16 and the immunoglobulin-containing sample may be combined within any suitable well-plate or vessel configuration and are allowed to incubate for an amount of time sufficient to form tagged antigen-antibody complexes 20, e.g., from 1 hour to 24 hours.

Once formed, in one embodiment, the tagged antigen-antibody complexes 20 may be recovered to enable downstream cleavage of the mass tags 12 from the complexes 20. The tagged antigen-antibody complexes 20 may be recovered from solution by any suitable method known in the art. In a particular embodiment where the antibody comprises an IgG antibody, the complexes may be biotinylated to attach biotin tags to the complexes 20. In one embodiment, as shown in FIG. 3, biotin tags may be attached to the complexes 20 via a biotin-tagged Protein A/G 22 (in liquid phase) comprising a biotin tag 24 and Protein A/G 26. Protein A/G is a recombinant fusion protein that combines IgG binding domains of both Protein A and Protein G. Protein A/G contains four Fc binding domains from Protein A and two from Protein G.

The biotin-tagged Protein A/G 22 may attach biotin tags 24 to substantially all or to all antibodies 18 in the complexes 20 to form biotin-tagged complexes 28 as shown in FIG. 3. The complexes 20 and the biotin-tagged Protein A/G 22 may be combined within any suitable well-plate or vessel configuration and are allowed to incubate for an amount of time sufficient for the biotin-tagged Protein A/G 22 to attach biotin tags 24 to at least substantially all of the complexes 20 to form the biotin-tagged complexes 28, e.g., from 1 hour to 24 hours.

Thereafter, in one embodiment, the biotin-tagged complexes 28 may be recovered from solution via their biotin tags. In an embodiment, the biotin-tagged complexes 28 may be captured via a solid phase. In one embodiment, the solid phase comprises streptavidin-coated magnetic beads 30, which are added to the solution containing the biotin-tagged complexes 28. The streptavidin-coated magnetic beads 30 are designed to capture biotinylated biopolymers such as antibodies, proteins and peptides. Applications where these beads have been used include bead-based assays such as standard ELISA and hybridoma screening of soluble antigens. Streptavidin-coated magnetic beads 30 are commercially available from a number of sources, e.g., from Applied Biosystems, Foster City, Calif., which sells such beads 30 under the name FMAT® Streptavidin Beads. The FMAT® Streptavidin Beads comprises a 1.0 mL solution with 6-8 micron beads, 0.5% w/v, for example.

For a description of the use of biotin tags and streptavidin-coated magnetic beads to recover antibody-containing components, see e.g., Kalle W H, Hazekamp-van Dokkum A M, Lohman P H, Natarajan A T, van Zeeland A A, Mullenders L H, The use of streptavidin-coated magnetic beads and biotinylated antibodies to investigate induction and repair of DNA damage: analysis of repair patches in specific sequences of uv-irradiated human fibroblasts. Anal Biochem. 1993 Feb. 1; 208(2):228-36.

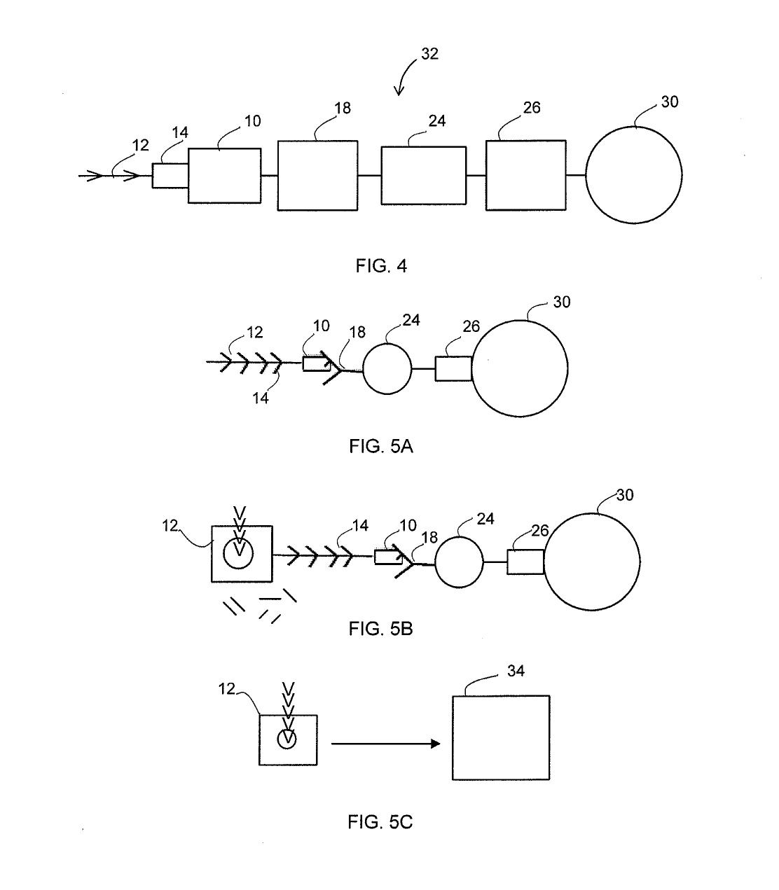

The streptavidin-coated magnetic beads 30 may be added to the solution containing the biotin-tagged complexes 28 within any suitable well-plate or vessel configuration and may be allowed to incubate for an amount of time sufficient for the streptavidin-coated magnetic beads 30 to bind to the biotin-tagged complexes 28 by their biotin tags 24 to form antibody-bound magnetic beads 32, e.g. from 1 hour to 24 hours. Once equilibrium has been reached, the magnetic beads 30 comprising bound complexes 28 (and possibly any free antibodies) may be removed from the solution. FIG. 4 depicts an exemplary antibody-bound magnetic bead 30 comprising a magnetic bead 32 bound to a biotin complex 28.

To free the mass tags 12 for detection by mass spectrometric analysis, the magnetic beads 32 (containing the biotin-tagged complexes 28) may be treated to free the mass tags 12 from the remainder of the complexes 28. In one embodiment, chemical or physical methods are used to cleave one or more bonds in the linker 14, resulting in the liberation of a respective mass tag 12. For example, the linker 14 may be cleavable by acid, base, chemical oxidation, chemical reduction, the catalytic activity of an enzyme, electrochemical oxidation or reduction, elevated temperature, photolysis, and thiol exchange. One skilled in the art would readily appreciate that the conditions under which the cleavage of the linker 14 will take place may be dependent on the structure and design of the linker 14.

In one embodiment, the linker 14 is cleavable by chemical methods and the antibody-bound magnetic beads 30 (containing the biotin-tagged complexes 28) may be added to a cleaver solution to free the mass tags 12 for detection by mass spectrometric analysis. The cleaver solution may be of any suitable pH or may include any suitable reducing agent that results in the majority of the mass tags 12 being removed from the attached complexes 28. In one embodiment, the linker 14 comprises a pH sensitive bond, such as an acyloxyalkyl ether, acetal, thioacetal, aminal, or imine bond, and is added to a solution at a pH to cleave the bond between the linker 14 and a respective mass tag 12.

In another embodiment, the linker 14 comprises a photolabile linker and cleavage of photolabile linker may be performed with UV light at a suitable wavelength, e.g., 350 nm, at intensities and times known to those in the art. The mass tags 12 may be freed by subjecting the antibody-bound magnetic beads 32 to UV light at a suitable wavelength, e.g., 250-364 nm, suitable to cleave the linkers 14.

In another embodiment, when the linker 14 comprises a linker cleavable by enzymatic cleavage, the cleavage of the linker 14 may be accomplished using enzymatic cleavage agents under suitable conditions. Examples of enzymatic cleavage agents include esterases which will cleave ester bonds, nucleases which will cleave phosphodiester bonds, proteases which cleave peptide bonds, and the like.

After the linkers 14 have been cleaved to free the mass tags 12 for detection by mass spectrometric analysis, the remaining portion of the antibody-bound magnetic beads 32 may be removed from the cleaver solution. The removal of the magnetic beads 32 (minus the mass tags 12) leaves a solution which principally comprises the mass tags 12. The mass tags 12 have a substantially lower molecular weight than labeled antigen-antibody complexes or labeled antibodies. In this way, the present invention provides a substantially simpler method of characterizing antibodies in a sample.

In one embodiment, the mass tags 12 are then transferred from the cleaver solution to a target plate for preparation for analysis by mass-spectrometry. Typically, target plates are dried, introduced into the mass spectrometer and irradiated. In one embodiment, the target plate comprises a Nanostructure Initiator Mass Spectrometry (NIMS) surface. NIMS is a desorption/ionization strategy for mass spectrometry that has been developed based on desorption/ionization from nanostructured surfaces. Overall, NIMS permits analysis of a wide range of molecules, is easily automated, and enables particularly high sensitivity.

Thereafter, the mass tags 12 are analyzed using mass spectrometry. As a first step, the mass tags 12 are ionized by any method known to one skilled in the art. These methods include, but are not limited to, electron ionization, chemical ionization, fast atom bombardment, field desorption, and matrix-assisted laser desorption ionization (“MALDI”), surface enhanced laser desorption ionization (“SELDI”), photon ionization, electrospray, and inductively coupled plasma. One skilled in the art will readily appreciate that the choice of ionization method can be determined based on the analyte to be measured, the type of sample, the type of detector, the choice of positive versus negative mode, etc. See U.S. Pat. Nos. 4,121,099; 4,137,750; 4,328,420; 4,963,736; 5,179,278; 5,248,875; 5,412,208; and 5,847,386, for example.

The mass analyzer of the mass spectrometer may similarly be selected to arrive at a desired lower limit of quantification (LLOQ), signal to noise ratio, and limit of detection. The mass analyzer may be a time of flight (TOF), quadrupole time of flight (Q-TOF), ion trap (IT), quadrupole ion trap (Q-IT), triple quadrupole (QQQ), Time-Of-Flight/Time-Of-Flight (TOFTOF), Orbitrap, or Fourier transform ion cyclotron resonance (FTICR) mass analyzer. In one embodiment, the mass analyzer is a TOF mass analyzer. The mass spectrometer may be a single stage or tandem mass spectrometer.

Once the samples are analyzed by the mass spectrometer, the mass spectrometer provides the user with a mass spectrum, e.g., the relative abundance of each m/z over a given range (e.g., 100 to 900). The results of an analyte assay (e.g., a mass spectrum) may be related to the amount of the analyte in the original sample by numerous methods known in the art. For example, given that sampling and analysis parameters are carefully controlled, the relative abundance of a given ion can be compared to a table that converts that relative abundance to an absolute amount of the original molecule. Alternatively, molecular standards can be run with the samples, and a standard curve constructed based on ions generated from those standards. Using such a standard curve, the relative abundance of a given ion can be converted back into an absolute amount of the original molecule. Numerous other methods for relating the presence or amount of an ion to the presence or amount of the original molecule are well known to those of ordinary skill in the art. Software for collection and handling of mass spectral data is available from numerous commercial sources, e.g., Pirouette® for Windows software available from Infometrix, Inc., Bothell, Wash. Utilizing the mass spectral data, by known methods, the presence and an amount of antibody in a particular sample may be correlated with the presence and the amount of the mass tags (which were tagged to antigens specific to the subject antibody) in the sample.

FIGS. 5A-5C provide a summary of the attachment and release of the different components within one embodiment of a method in accordance with the present invention. FIG. 5A shows all the components necessary to form an exemplary antibody-bound magnetic bead 32 as described above in accordance with one aspect of the present invention. It is understood that one of each component, e.g., antibody-bound magnetic bead 32, is shown for ease of reference, though multiple antibody-bound magnetic beads 32 are formed. As shown, a mass tag 12 is bonded to an antigen 10, which is complexed with an antibody 18 to form a tagged antibody-antigen complex 20. The antibody 18 is then bound to a biotin-tagged Protein A/G 22 comprising a biotin tag 24 and Protein A/G 26 to form a biotin-tagged complex 20. A streptavidin-coated magnetic bead 30 is utilized to capture the biotin-tagged complex 28 via the biotin tag 24 on the complex 28. Thereafter, the magnetic bead 30 comprising the biotin-tagged complex 28 is released into a cleaver solution and linker 14 is cleaved to free the mass tag 12. As shown in FIG. 5B, the linker 14 is cleaved leaving the mass tag 12 and the remaining portion of the biotin-tagged complex 28 and the magnetic bead 30. The antibody-bound magnetic bead 32 (minus the mass tag 12) may be removed and the mass tag 12 may be transferred to a target plate for preparation for analysis by mass-spectrometry, e.g. a NIMS surface. Thereafter, the mass tag 12 is analyzed via the mass spectrometer 34 to give both qualitative and quantitative information. The presence and amount of the mass tag 12 may be determined and correlated with an amount of a particular antibody 18 in the sample.

In accordance with another aspect of the present invention, there is provided a kit for carrying out the above-described methods. In one embodiment, there is provided a kit for use in mass spectrometric analysis. The kit comprises (a) a plurality of antigens 10; (b) a plurality of mass tags 12 for bonding (covalent or non-covalent) to respective ones of the plurality of antigens 10 to produce a plurality of tagged antigens 16; (c) a plurality of antibodies 18 for complexing with respective ones of the plurality of tagged antigens 16 to form a plurality of tagged antigen-antibody complexes 20 and/or instructions for adding a sample comprising a plurality of antibodies 18 to the plurality of tagged antigens 16; and (d) instructions and/or reagents for cleaving the mass tags 12 from the plurality of tagged antigen-antibody complexes 20. Optionally, the kit may further comprise one or more of instructions for analyzing the mass tags 12 by mass spectrometry, quality control specimens, a solid phase 30 for removing the tagged antigen-antibody complexes 20 from solution, and tags 24 for attaching the tagged antigen-antibody complexes 20 to the solid phase. In an alternative embodiment, the kit comprises a plurality of antibodies 18 that are added to a plurality of respective antigens 10 prior to tagging the plurality of antigens 10 with mass tags 12 and/or instructions for adding a plurality of antibodies 18 to a plurality of respective antigens 10 prior to tagging the plurality of antigens 10 with mass tags 12.

The teachings of the references cited throughout the specification are incorporated herein in their entirety by this reference to the extent they are not inconsistent with the teachings herein. It should be understood that the examples and the embodiments described herein are for illustrative purposes only and that various modifications or changes in light thereof will be suggested to persons skilled in the art and are to be included within the spirit and purview of this application.

The invention claimed is:

1. A method for the characterization or detection of one or more antibodies in a sample comprising:
(a) obtaining a tagged antigen comprising an antigen and a mass tag attached to the antigen, said tagged antigen having specificity for the antibody;
(b) combining the tagged antigen with the antibody in the sample to form a tagged antigen-antibody complex;
(c) cleaving the mass tag from the tagged antigen-antibody complex; and
(d) analyzing the mass tag via a mass spectrometer to determine a presence of mass tag; and, optionally,
(e) correlating the presence of the mass tag with a presence of the antibody in the sample.

2. The method of claim 1, wherein the analyzing further comprises quantifying an amount of the mass tag in the sample, and wherein the correlating further comprises correlating the amount of the mass tag with an amount of the antibody.

3. The method of claim 1, further comprising capturing the tagged antigen-antibody complex via tagging the antibody of the antigen-antibody complex with a biotin tag to form a biotin-tagged complex and capturing the biotin-tagged complex via a streptavidin-coated magnetic bead.

4. The method of claim 1, wherein the mass tag is covalently bonded to the antigen via a linker, and wherein the linker is cleavable by at least one of chemical, photolytic, or enzymatic methods.

5. A kit for the characterization or detection of one or more immunoglobulins in a sample comprising:
(a) a first container comprising a first antigen linked with a first mass tag;
(b) a second container comprising a second antigen linked with a second mass tag; and
(c) optionally, instructions for adding said first and/or second antigen to a sample comprising a plurality of antibodies.

6. The kit of claim 5, wherein the kit further comprises at least one of instructions for analyzing the mass tags by mass spectrometry and quality control specimens.

7. The kit of claim 5, wherein the kit further comprises a solid phase for removing tagged antigen-antibody complexes from a solution.

8. The kit of claim 5, wherein the kit further comprises instructions and/or reagents for cleaving the first and second mass tags from the first and second antigens, respectively.

9. The kit of claim 5, wherein the kit further comprises a container of biotin.

10. The kit of claim 5, wherein the first antigen is linked to the first mass tag by a first linker and the second antigen is linked to the second mass tag by a second linker.

11. The kit of claim 10, wherein said first linker and said second linker are the same or different.

12. The kit of claim 11, wherein said first linker is cleavable by a first means and said second linker is cleavable by a second means different from said first means.

13. The kit of claim 5, wherein said first antigen and or said second antigen is a disease or disorder biomarker.

14. The kit of claim 5, wherein said first antigen and/or said second antigen is a secondary antibody.

15. The kit of claim 5, wherein said kit further comprises a first solid support having a first secondary antibody bound thereto, wherein said first secondary antibody specifically binds to said first antigen; and a separate second solid support having a second secondary antibody bound thereto, wherein said second secondary antibody specifically binds to said second antigen.

16. The kit of claim 5, wherein said kit further comprises a solid support comprising a first portion having a first secondary antibody bound thereto, wherein said first secondary antibody specifically binds to said first antigen; and a second portion having a second secondary antibody bound thereto, wherein said second secondary antibody specifically binds to said second antigen.

17. A composition useful for characterizing or detecting one or more immunoglobulins in a sample, said composition comprising at least one antigen that binds to at least one of said one or more immunoglobulins; and at least one mass tag associated with said at least one antigen via a cleavable linker.

18. The composition of claim 17, wherein said at least one antigen is an anti-idiotype antibody.

19. The composition of claim 17, wherein said at least one antigen is a secondary antibody.

20. The composition of claim 17, wherein said at least one antigen is a biomarker for a disease or disorder.

21. The method of claim 1, wherein said method further comprises separating mass tag from antigen-antibody complex after said cleaving step; and capturing mass tag onto a NIMS surface prior to said analyzing step.

22. The method of claim 21, wherein said mass tag is a fluorous mass tag.

23. The method of claim 1, wherein said obtaining step comprises obtaining a first tagged antigen linked with a first mass tag and a second tagged antigen linked with a second mass tag; said combining step comprises incubating said first and second tagged antigens in said sample to allow said first and second tagged antigens to form a first tagged antigen-antibody complex and a second tagged antigen-antibody complex; said cleaving step comprises cleaving said first mass tag from said first tagged antigen-antibody complex and cleaving said second mass tag from said second tagged antigen-antibody complex; and said analyzing step comprises analyzing the first and second mass tag via a mass spectrometer to determine a presence of the first and second mass tag in the sample.

24. A method for the characterization or detection of two or more antibodies in a sample comprising:
(a) incubating a first tagged antigen linked with a first mass tag and a second tagged antigen linked with a second mass tag in the sample under conditions to form a first tagged antigen-antibody complex and a second tagged antigen-antibody complex;
(b) separating said first tagged antigen-antibody complex and said second tagged antigen-antibody complex from said sample;
(c) cleaving said first and second mass tags from said first and second tagged antigen-antibody complexes; and
(d) analyzing the first and second mass tags via a mass spectrometer to determine a presence of first and second mass tag.

25. The method of claim 25, wherein, after cleaving step, the method further comprises capturing onto a NIMS surface said first and second mass tags cleaved from said first and second tagged antigen-antibody complexes.
